(19) United States
(12) Patent Application Publication
(10) Pub. No.: US 2023/0051006 A1
(43) Pub. Date: Feb. 16, 2023

(54) NOTIFICATION OF PRIVACY ASPECTS OF HEALTHCARE PROVIDER ENVIRONMENTS DURING TELEMEDICINE SESSIONS

(71) Applicant: Optum, Inc., Minnetonka, MN (US)

(72) Inventors: Ramprasad Anandam Gaddam, Mumbai (IN); Kristine Xu, Stoneham, MA (US); Gregory J. Boss, Saginaw, MI (US); Jon Kevin Muse, Thompsons Station, TN (US)

(21) Appl. No.: 17/444,880

(22) Filed: Aug. 11, 2021

(51) Int. Cl. G16H 10/60; G16H 40/67

(52) U.S. Cl. CPC G16H 10/60; G16H 40/67

(57) ABSTRACT

A method comprises obtaining a video stream of a first party who is engaging with a second party in a telemedicine session; determining statuses of one or more privacy aspects of a first party environment, wherein the privacy aspects of the first party environment are aspects of the first party environment that have a potential to compromise privacy of sensitive information provided by the second party to the first party during the telemedicine session; and causing a second party computing device to present a user interface of a telemedicine facilitation application, wherein the user interface of the telemedicine facilitation application includes the video stream of the first party and also includes a set of one or more notifications, wherein each of the one or more notifications indicates the status of a different one of the privacy aspects of the first party environment.

### BACKGROUND

Telemedicine sessions are an increasingly common way for patients to interact with healthcare providers. During a telemedicine session, a patient may communicate with a healthcare provider via a telephonic and/or video link. The patient may wish to communicate sensitive information to the healthcare provider during a telemedicine session. The sensitive information may include personal health information, personally identifying information, and other types of information. For example, the patient may need to describe a potentially embarrassing health condition or show the healthcare provider a private body part.

### SUMMARY

The present disclosure describes devices, systems, and methods for notifying patients of statuses of privacy aspects of healthcare provider environments during telemedicine sessions. As previously mentioned, a patient may wish to communicate sensitive information to a healthcare provider during a telemedicine session. However, the patient may be unwilling to communicate the sensitive information to the healthcare provider during the telemedicine session if the patient is concerned that the sensitive information will be obtained by an unauthorized person. For example, if the healthcare provider is working from home, the patient may be concerned that a family member of the healthcare provider may see or overhear the sensitive information during the telemedicine session. In another example, the patient may be concerned that there may be a smart speaker or other audio device in an environment of the healthcare provider that may overhear the sensitive information and send the sensitive information out of the environment of the healthcare provider during the telemedicine session. In another example, the patient may be concerned that other patients or unauthorized personnel in an office of the healthcare provider may see or overhear the sensitive information during the telemedicine session.

There are several technical problems associated with conventional systems for controlling access to sensitive information provided during telemedicine sessions. For example, in conventional systems, the patient is not informed of statuses of privacy aspects of the environment of the healthcare provider (i.e., the healthcare provider environment) during a telemedicine session. Moreover, the patient is not informed when statuses of privacy aspects of the healthcare provider environment change during the course of the telemedicine session. Informing the patient of statuses of current privacy aspects of the healthcare provider environment may help the patient feel more at ease disclosing the sensitive information to the healthcare provider. Providing an efficient user interface that informs the patient of statuses of the privacy aspects of the healthcare provider environment may make telemedicine sessions more efficient, which may conserve network bandwidth. Moreover, providing an efficient user interface may increase the efficiency of an application facilitating the telemedicine session by enabling the patient to access relevant statuses of the privacy aspects of the healthcare provider environment without the patient needing training or instruction on how to determine the statuses of privacy aspects of the healthcare provider environment.

As described in this disclosure, an application facilitating a telemedicine session may display statuses of privacy aspects of a healthcare provider environment in a user interface of the telemedicine session. The user interface of the telemedicine session also includes a video feed of the healthcare provider. The user interface may indicate statuses of one or more of the privacy aspects. The application may update the status indication of a privacy aspect if there is a change in the status of the privacy aspect and update the user interface accordingly. Because the statuses of one or more privacy aspects are displayed in the same user interface as the video feed of the healthcare provider, the computing device of the patient may be configured to generate and output the user interface to efficiently present information about the privacy aspects of the healthcare provider environment and receive user input without requiring the patient to have training or experience navigating the user interface of the telemedicine session.

In some examples, the application may generate notifications of changes to statuses of the privacy aspects of the healthcare provider environment in addition to, or as an alternative to, presenting the statuses of the privacy aspects of the healthcare provider environment. Generating notifications whenever a status of a privacy aspect of the healthcare provider environment changes may be distracting to the patient and may diminish the effectiveness of the efficiency of the telemedicine session. For example, the patient might not find it helpful for the user interface of the telemedicine session to display a notification whenever a status of a privacy aspect of the healthcare provider environment changes when the patient is not providing sensitive information to the healthcare provider. Ensuring that a computing system that provides an efficient user interface for notifying the patient of changes to statuses of privacy aspects of the healthcare provider environment without overburdening the patient with notifications is therefore a technical challenge.

In accordance with one or more examples of this disclosure, the computing system may determine a stress level of the patient based on audio and video data of the telemedicine session. The computing system may determine whether to generate a notification based on the stress level of the patient. In general, the patient's stress level rises when disclosing or preparing to disclose sensitive information. If the patient exhibits lower stress levels, the computing system may make a determination not to notify the patient of a change to a status of a privacy aspect of the healthcare provider environment. However, if the patient exhibits a high stress level, the computing system may make a determination to notify the patient of a change to the status of the same privacy aspect of the healthcare provider environment.

In one example, this disclosure describes a method comprising: obtaining, by a computing system, a video stream of a first party who is engaging with a second party in a telemedicine session; determining, by the computing system, at the start of and/or during the telemedicine session, statuses of one or more privacy aspects of a first party environment, wherein the first party environment is an environment of the first party, wherein the privacy aspects of the first party environment are aspects of the first party environment that have a potential to compromise privacy of sensitive information provided by the second party to the first party during the telemedicine session; and causing, by the computing system, a second party computing device to present a user interface of a telemedicine facilitation application, wherein the user interface of the telemedicine facilitation application includes the video stream of the first party and also includes a set of one or more notifications, wherein each of the one or more notifications indicates the status of a different one of the privacy aspects of the first party environment.

In another example, this disclosure describes a computing system comprising: a communication unit configured to obtain a video stream of a first party who is engaging with a second party in a telemedicine session; and one or more processors implemented in circuitry and in communication with the memory, the one or more processors configured to: determine, at the start of and/or during the telemedicine session, statuses of one or more privacy aspects of a first party environment, wherein the first party environment is an environment of the first party, wherein the privacy aspects of the first party environment are aspects of the first party environment that have a potential to compromise privacy of sensitive information provided by the second party to the first party during the telemedicine session; and cause a second party computing device to present a user interface of a telemedicine facilitation application, wherein the user interface of the telemedicine facilitation application includes the video stream of the first party and also includes a set of one or more notifications, wherein each of the one or more notifications indicates the status of a different one of the privacy aspects of the first party environment.

In another example, this disclosure describes a non-transitory computer-readable medium having instructions stored thereon that, when executed, cause one or more processors to: obtain a video stream of a first party who is engaging with a second party in a telemedicine session; determine, at the start of and/or during the telemedicine session, statuses of one or more privacy aspects of a first party environment, wherein the first party environment is an environment of the first party, wherein the privacy aspects of the first party environment are aspects of the first party environment that have a potential to compromise privacy of sensitive information provided by the second party to the first party during the telemedicine session; and cause a second party computing device to present a user interface of a telemedicine facilitation application, wherein the user interface of the telemedicine facilitation application includes the video stream of the first party and also includes a set of one or more notifications, wherein each of the one or more notifications indicates the status of a different one of the privacy aspects of the first party environment.

The details of one or more aspects of the disclosure are set forth in the accompanying drawings and the description below. Other features, objects, and advantages of the techniques described in this disclosure will be apparent from the description, drawings, and claims.

### BRIEF DESCRIPTION OF THE DRAWINGS

FIG. 1 is a block diagram illustrating an example system in accordance with one or more aspects of this disclosure.

FIG. 2 is a block diagram illustrating an example computing system that implements a telemedicine facilitation application in accordance with one or more aspects of this disclosure.

FIG. 3 is a conceptual diagram illustrating example notifications in accordance with one or more aspects of this disclosure.

FIG. 4 is a conceptual diagram illustrating example notifications in accordance with one or more aspects of this disclosure.

FIG. 5 is a flowchart illustrating an example operation of a telemedicine facilitation application in accordance with one or more aspects of this disclosure.

FIG. 6 is a flowchart illustrating an example operation in which notifications are displayed dependent on stress levels, in accordance with one or more aspects of this disclosure.

### DETAILED DESCRIPTION

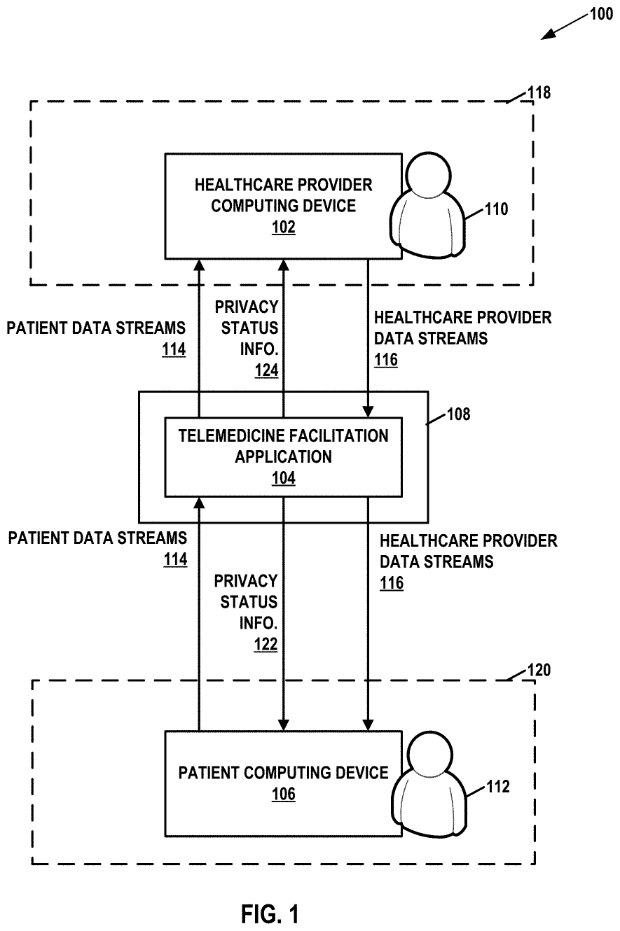

FIG. 1 is a block diagram illustrating an example system 100 in accordance with one or more aspects of this disclosure. In the example of FIG. 1, system 100 includes a healthcare provider computing device 102, a telemedicine facilitation application 104, and a patient computing device 106. Telemedicine facilitation application 104 may operate on one or more of a computing system 108, healthcare provider computing device 102, or patient computing device 106. A healthcare provider 110 uses healthcare provider computing device 102. A patient 112 uses patient computing device 106. In other examples, system 100 may include more, fewer, or different components. For instance, computing system 100 may include multiple healthcare provider computing devices, patient computing devices, and so on. Computing system 108 may include one or more computing devices. In examples where computing system 108 includes two or more computing devices, the computing devices of computing system 108 may act together as a system. Example types of computing devices include server devices, personal computers, mobile devices (e.g., smartphones, tablet computers, wearable devices), intermediate network devices, and so on.

Healthcare provider 110 may be a person who provides healthcare services. For example, healthcare provider 110 may be a doctor, a nurse, a medical staff member (including, e.g., a medical scheduling clerk, a physician assistant, a lab technician, etc.), a mental health therapist, a chiropractor, an optometrist, a dentist, a clinician, or another type of person who provides healthcare services. Patient 112 may be a person receiving healthcare services. For example, patient 112 may be receiving healthcare services related to a suspected infection, heart condition, skin condition, cancer, injury, and so on. In some examples, patient 112 may be accompanied by another person, such as a parent or guardian.

Healthcare provider 110 and patient 112 may use healthcare provider computing device 102 and patient computing device 106, respectively, to engage in a telemedicine session facilitated by telemedicine facilitation application 104. Telemedicine sessions are useful in a variety of circumstances. For example, telemedicine sessions may be useful to assess minor patient health complaints, triage patients, discuss lab results, discuss upcoming in-person healthcare appointments, and so on. Telemedicine sessions have become especially common for routine health check-ins. Telemedicine sessions may be especially useful for patients or healthcare providers who are located in rural or otherwise remote areas.

During a telemedicine session, telemedicine facilitation application 104 may obtain one or more patient data streams 114, e.g., from patient computing device 106 and/or one or more other computing devices associated with patient 112. In the example of FIG. 1, patient computing device 106 may transmit one or more of patient data streams 114 to telemedicine facilitation application 104, which may forward one or more of patient data streams 114 to healthcare provider computing device 102. In some examples, telemedicine facilitation application 104 may modify one or more of patient data streams 114 prior to forwarding one or more of patient data streams 114 to healthcare provider computing device 102. Patient data streams 114 may include an audio stream that includes audio data representing sound captured at a location of patient 112, e.g., by a microphone of patient computing device 106. Patient data streams 114 may include a video stream that includes video data representing a visual scene captured at the location of patient 112, e.g., by a video camera of patient computing device 106. In some examples, patient computing device 106 may transmit one or more of patient data streams 114 directly to healthcare provider computing device 102.

Similarly, during a telemedicine session, telemedicine facilitation application 104 may obtain one or more healthcare provider data streams 116, e.g., from healthcare provider computing device 102 and/or one or more other computing devices associated with healthcare provider 110. In the example of FIG. 1, healthcare provider computing device 102 may transmit healthcare provider data streams 116 to telemedicine facilitation application 104, which may forward healthcare provider data streams 116 to patient computing device 106. In some examples, telemedicine facilitation application 104 may modify healthcare provider data streams 116 prior to forwarding one or more of healthcare provider data streams 116 to patient computing device 106. Healthcare provider data streams 116 may include an audio stream that includes audio data representing sound captured at a location of healthcare provider 110, e.g., by a microphone of healthcare provider computing device 102. Healthcare provider data streams 116 may include a video stream that includes video data representing a visual scene captured at the location of healthcare provider 110, e.g., by a video camera of healthcare provider computing device 102. In some examples, healthcare provider computing device 102 may transmit one or more of healthcare provider data streams 116 directly to patient computing device 106.

At the start of and/or during a telemedicine session, patient 112 may need to provide sensitive information to healthcare provider 110. For example, patient 112 may need to describe health conditions of patient 112, drug use, sexual history, domestic relationship issues, abuse history, mental health issues, vital statistics, infection status, pregnancy status, and so on. In some instances, patient 112 may need to show a private body part, skin condition, injury, or visually provide other information to healthcare provider 110. Likewise, at the start of and/or during a telemedicine session, healthcare provider 110 may need to provide sensitive information to patient 112. For instance, healthcare provider 110 may need to inform patient 112 of a diagnosis or test result that patient 112 may wish to be kept secret.

Patient 112 may only want the sensitive information to be disclosed to people, such as healthcare provider 110, who are obligated to maintain the secrecy of the sensitive information. For example, patient 112 may not want the sensitive information to be disclosed to family members of healthcare provider 110, family members of patient 112, other patients of healthcare provider 110, or to random passersby. While telemedicine sessions are useful, telemedicine sessions may diminish the ability of patient 112 to assess the risks that sensitive information may be disclosed to other people. For example, in a telemedicine session, patient 112 may only be able to see what is in the field of view of a camera of healthcare provider computing device 102. Thus, in this example, patient 112 may not be able to determine whether there are one or more people present in healthcare provider environment 118 who are off camera. This problem may be especially acute if healthcare provider computing device 102 is using an artificial background. Likewise, patient 112 may have a diminished ability to assess whether people in healthcare provider environment 118 may overhear sensitive information. For instance, patient 112 may not be able to determine the volume level of healthcare provider computing device 102, determine whether voices can be heard through the walls of healthcare provider environment 118, determine masking noise levels in healthcare provider environment 118, and so on.

Moreover, patient 112 may not be able to determine whether there are devices in healthcare provider environment 118 that may receive the sensitive information. For example, smart speaker devices, security cameras, smartphones, Internet of Things (IoT) devices, and other types of devices may detect sounds and visual scenes in healthcare provider environment 118. For instance, a smart speaker device may constantly be detecting sounds in healthcare provider environment 118 and sending audio data representing the sounds to a remote computing device for processing, e.g., to detect commands directed to the smart speaker device. However, such audio data may be intercepted or may be listened to by personnel of a provider of the smart speaker device.

Similar considerations may apply with respect to healthcare provider 110 providing sensitive information to patient 112. In other words, healthcare provider 110 may not want to provide sensitive information to patient 112 if specific people or devices are present in patient environment 120. For example, healthcare provider 110 not want to provide information about physical abuse to patient 112 if an abuser of patient 112 is present in patient environment 120 because doing so may jeopardize the safety of patient 112.

As previously described, conventional telemedicine facilitation applications do not provide efficient, or any, user interfaces that inform patient 112 of statuses of privacy aspects of healthcare provider environment 118. Likewise, conventional telemedicine facilitation applications do not provide efficient, or any user interfaces that inform healthcare provider 110 of statuses of privacy aspects of patient environment 120.

Techniques of this disclosure may address this problem. For instance, in accordance with one or more techniques of this disclosure, telemedicine facilitation application 104 may obtain a video stream (e.g., one of healthcare provider data streams 116) of healthcare provider 110 who is engaging with patient 112 in a telemedicine session. Telemedicine facilitation application 104 may determine at the start of and/or during the telemedicine session, statuses of one or more privacy aspects of healthcare provider environment 118. Healthcare provider environment 118 is an environment of healthcare provider 110. The privacy aspects of healthcare provider environment 118 are aspects of healthcare provider environment 118 that have a potential to compromise privacy of sensitive information provided by patient 112 to healthcare provider 110 during the telemedicine session. Furthermore, telemedicine facilitation application 104 may cause patient computing device 106 to present a user interface of telemedicine facilitation application 104. The user interface of telemedicine facilitation application 104 includes the video stream of healthcare provider 110 and may also include a set of one or more notifications. Each of the one or more notifications may indicate the status of a different one of the privacy aspects of healthcare provider environment 118. In the example of FIG. 1, telemedicine facilitation application 104 may send privacy status information 122 specifying the notifications to patient computing device 106.

In accordance with one or more techniques of this disclosure, telemedicine facilitation application 104 may obtain a video stream (e.g., one of patient data streams 114) of patient 112 who is engaging with healthcare provider 110 in a telemedicine session. Telemedicine facilitation application 104 may determine, at the start of and/or during the telemedicine session, statuses of one or more privacy aspects of patient environment 120. Patient environment 120 is an environment of patient 112. The privacy aspects of patient environment 120 are aspects of patient environment 120 that have a potential to compromise privacy of sensitive information provided by healthcare provider 110 to patient 112 during the telemedicine session. Furthermore, telemedicine facilitation application 104 may cause healthcare provider computing device 102 to present a user interface of telemedicine facilitation application 104. The user interface of telemedicine facilitation application 104 includes the video stream of patient 112 and may also include a set of one or more notifications. Each of the one or more notifications may indicate the status of a different one of the privacy aspects of patient environment 120. In the example of FIG. 1, telemedicine facilitation application 104 may send privacy status information 124 specifying the notifications to patient computing device 106.

Thus, in a more general example, telemedicine facilitation application 104 may obtain a video stream (e.g., one of patient data streams 114 or healthcare provider data streams 116) of a first party (e.g., patient 112 or healthcare provider 110) who is engaging with a second party (e.g., patient 112 or healthcare provider 110) in a telemedicine session. Telemedicine facilitation application 104 may determine, at the start of and/or during the telemedicine session, statuses of one or more privacy aspects of a first-party environment (e.g., patient environment 120 or healthcare provider environment 118). The first-party environment is an environment of the first party. The privacy aspects of the first-party environment are aspects of the first-party environment that have a potential to compromise privacy of sensitive information provided by the second party to the first party during the telemedicine session. Furthermore, telemedicine facilitation application 104 may cause a second-party computing device to present a user interface of telemedicine facilitation application 104. The user interface of telemedicine facilitation application 104 includes the video stream of the first party and may also include a set of one or more notifications. Each of the one or more notifications may indicate the status of a different one of the privacy aspects of the first-party environment.

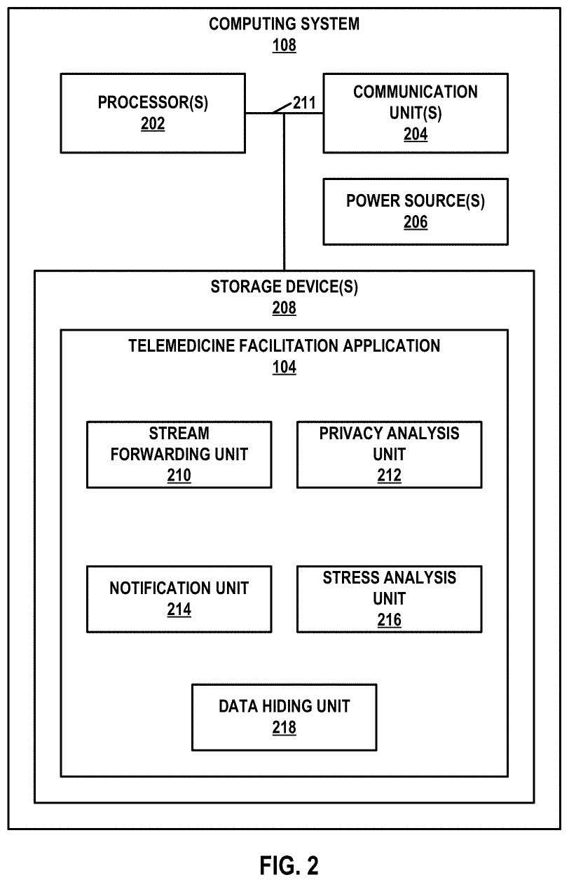

FIG. 2 is a block diagram illustrating example components of computing system 108 in accordance with one or more aspects of this disclosure. FIG. 2 illustrates only one example of computing system 108, without limitation on many other example configurations of computing system 108. Computing system 108 may be the same as healthcare provider computing device 102, patient computing device 106, or may comprise a separate system of one or more computing devices.

As shown in the example of FIG. 2, computing system 108 includes one or more processors 202, one or more communication units 204, one or more power sources 206, one or more storage devices 208, and one or more communication channels 211. Computing system 108 may include other components. For example, computing system 108 may include input devices, output devices, display screens, and so on. Communication channel(s) 210 may interconnect each of processor(s) 202, communication unit(s) 204, and storage device(s) 208 for inter-component communications (physically, communicatively, and/or operatively). In some examples, communication channel(s) 210 may include a system bus, a network connection, an inter-process communication data structure, or any other method for communicating data. Power source(s) 206 may provide electrical energy to processor(s) 202, communication unit(s) 204, storage device(s) 206 and communication channel(s) 210. Storage device(s) 208 may store information required for use during operation of computing system 108.

Processor(s) 202 comprise circuitry configured to perform processing functions. For instance, one or more of processor(s) 202 may be a microprocessor, an application-specific integrated circuit (ASIC), a field-programmable gate array (FPGA), or another type of processing circuitry. In some examples, processor(s) 202 of computing system 108 may read and execute instructions stored by storage device(s) 208. Processor(s) 202 may include fixed-function processors and/or programmable processors. Processor(s) 202 may be included in a single device or distributed among multiple devices.

Communication unit(s) 204 may enable computing system 108 to send data to and receive data from one or more other computing devices (e.g., via a communications network, such as a local area network or the Internet). In some examples, communication unit(s) 204 may include wireless transmitters and receivers that enable computing system 108 to communicate wirelessly with other computing devices. Examples of communication unit(s) 204 may include network interface cards, Ethernet cards, optical transceivers, radio frequency transceivers, or other types of devices that are able to send and receive information. Other examples of such communication units may include BLUETOOTH™, 3G, 4G, 5G, and WI-FI™ radios, Universal Serial Bus (USB) interfaces, etc. Computing system 108 may use communication unit(s) 204 to communicate with one or more other computing devices or systems, such as client device 104. Communication unit(s) 204 may be included in a single device or distributed among multiple devices.

Processor(s) 202 may read instructions from storage device(s) 208 and may execute instructions stored by storage device(s) 208. Execution of the instructions by processor(s) 202 may configure or cause computing system 108 to provide at least some of the functionality ascribed in this disclosure to computing system 108. Storage device(s) 208 may be included in a single device or distributed among multiple devices.

As shown in the example of FIG. 2, storage device(s) 208 may include computer-readable instructions associated with telemedicine facilitation application 104. In the example of FIG. 2, telemedicine facilitation application 104 may include a stream forwarding unit 210, a privacy analysis unit 212, a notification unit 214, a stress analysis unit 216, and a data hiding unit 218. In other examples, telemedicine facilitation application 104 may include more, fewer, or different units. Moreover, the units of telemedicine facilitation application 104 shown in the example of FIG. 2 are presented for purposes of explanation and may not necessarily correspond to actual software units or modules within telemedicine facilitation application 104.

Stream forwarding unit 210 may be configured to obtain data streams via communication unit(s) 204. The data streams may include patient data streams 114 and healthcare provider data streams 116. As discussed elsewhere in this disclosure, patient data streams 114 and healthcare provider data streams 116 may include audio and video streams. Stream forwarding unit 210 may forward healthcare provider data streams 116 from healthcare provider computing device 102 to patient computing device 106. Likewise, stream forwarding unit 210 may forward patient data streams 114 from patient computing device 106 to healthcare provider computing device 102. In some instances, telemedicine facilitation application 104 may modify the data streams before stream forwarding unit 210 forwards the data streams.

Privacy analysis unit 212 may be configured to determine, at the start of or during a telemedicine session, statuses of one or more privacy aspects of an environment of a party to the telemedicine session, e.g., healthcare provider environment 118 or patient environment 120. Privacy analysis unit 212 may determine the statuses of the privacy aspects of the environment of the party in one or more ways. For ease of explanation, this disclosure describes examples of determining status of privacy aspects of healthcare provider environment 118, but such examples may also apply, with appropriate changes, to determining statuses of the privacy aspects of patient environment 120.

In one example, healthcare provider data streams 116 may include data associated with devices in healthcare provider environment 118. In this example, healthcare provider computing device 102 may receive wireless communication signals from devices in healthcare provider environment 118. Such devices may include IoT devices, Internet of Medical Things (IoMT) devices, smart speakers, smartphones, personal computers, tablet computers, and so on. The wireless communication signals may be Bluetooth signals, WiFi signals, ZigBee signals, or other types of wireless signals. The wireless communication signals may include sufficient data for healthcare provider computing device 102 to identify device types of the devices. Privacy analysis unit 212 may determine, based on the data associated with devices in healthcare provider environment 118, whether healthcare provider environment 118 includes one or more devices that may pose a security concern. For example, privacy analysis unit 212 may determine, based on data indicating the types of devices in healthcare provider environment 118, that healthcare provider environment 118 includes one or more devices (e.g., smart speakers, etc.) that may pose a security concern.

In some examples, healthcare provider computing device 102 may use wireless locating techniques to estimate locations of devices in healthcare provider environment 118. The data associated with devices in healthcare provider environment 118 may include information regarding locations of devices in healthcare provider environment 118. For instance, healthcare provider computing device 102 may determine distances of devices in healthcare provider environment 118 based on wireless signal strengths of the devices in healthcare provider environment 118. In some examples, healthcare provider computing device 102 may determine directions of device in healthcare provider environment 118 based on various properties of wireless signals, such as delay of receipt between two or more antennas, direction of induced current, and so on.

In some examples, the data associated with devices in healthcare provider environment 118 may include data identifying individual devices. For instance, the example where the devices in healthcare provider environment 118 include a mobile phone, the data associated with the mobile phone may include a phone number of the mobile phone, Media Access Control (MAC) address of the mobile phone, or other data. Privacy analysis unit 212 or healthcare provider computing device 102 may map the data associated with the mobile phone to individual people or may determine that the mobile phone is associated with an unknown person. Thus, privacy analysis unit 212 may determine whether a particular person is likely to be in healthcare provider environment 118 or whether one or more unknown persons are likely present in healthcare provider environment 118.

In some examples where healthcare provider data streams 116 include a video stream, privacy analysis unit 212 may analyze the video stream to determine statuses of one or more privacy aspects of healthcare provider environment 118. For example, privacy analysis unit 212 may use facial recognition technology to determine whether people in a field of view of the video stream are authorized or not. The presence of unauthorized people in healthcare provider environment 118 may be a privacy aspect of healthcare provider environment 118. In some examples, privacy analysis unit 212 may determine information (e.g., roles, name, job titles, etc.) about people identified in the video stream. Privacy analysis unit 212 may access a database that includes the information about people to determine the information about the people identified in the video stream. A user interface of telemedicine facilitation application 104 may indicate the people and information about the people.

In some examples, privacy analysis unit 212 may determine the proximity and/or locations of people shown in the video stream. For instance, privacy analysis unit 212 may use disparity of images of people in stereoscopic video streams to determine proximity of people shown in the video streams. In such examples, privacy analysis unit 212 may determine that a person shown in the video stream is not in healthcare provider environment 118 if the person is sufficiently far away.

In some examples, privacy analysis unit 212 may analyze the video stream to identify types of devices or objects in healthcare provider environment 118 that correspond to privacy aspects. For instance, privacy analysis unit 212 may use image recognition technology to identify devices (e.g., smart speakers, cameras, microphones, IoT devices, etc.) that correspond to privacy aspects in healthcare provider environment 118. In some examples, privacy analysis unit 212 may analyze the video stream to identify objects (e.g., undraped windows, open doors, etc.) that may compromise privacy in healthcare provider environment 118. Furthermore, in some examples, privacy analysis unit 212 may attempt to verify devices identified in the video stream. For instance, privacy analysis unit 212 may request healthcare provider computing device 102 output a wireless request to a device identified in the video stream to identify itself.

In some examples where healthcare provider data streams 116 include an audio stream, privacy analysis unit 212 may analyze the audio stream to determine statuses of one or more privacy aspects of healthcare provider environment 118. For example, privacy analysis unit 212 may analyze the audio stream to determine whether there are voices other than a voice of healthcare provider 110 present in healthcare provider environment 118. Where such voices belong to people in other rooms (e.g., exam rooms), the sound of the voice of patient 112 may likewise be heard in other rooms. Therefore, the sound of other peoples' voices may indicate that the privacy of healthcare provider environment 118 may be compromised.

In some examples, privacy analysis unit 212 may determine an intelligibility metric for other voices in healthcare provider environment 118 (i.e., voices other than the voice of healthcare provider 110 or other known authorized person). The intelligibility metric may indicate how intelligible the other voices are. A low intelligibility metric may indicate that the other voices are less intelligible. Conversely, a high intelligibility metric may indicate that the other voices are more intelligible. Higher intelligibility metrics may correspond to situations in which the other voices are passing through walls or coming through open windows or doors. Hence, higher intelligibility metrics may correspond to situations in which the voice of patient 112 may also be heard by unauthorized people. In some examples, to determine the intelligibility metric, privacy analysis unit 212 may determine a number of intelligible words within a given time interval (e.g., 10 seconds). The privacy aspects of healthcare provider environment 118 may include audio privacy and privacy analysis unit 212 may determine that the status of this privacy aspect is compromised if the intelligibility metric is above a given threshold. For instance, privacy analysis unit 212 may award 2 points for each intelligible word in a 10 second time interval and determine that the status of this privacy aspect is compromised if there are 6 points awarded in the time interval.

In some examples, privacy analysis unit 212 may analyze the audio stream for voices of authorized and unauthorized people. Authorized people may include people who are not a privacy risk for patient 112. For example, privacy analysis unit 212 may analyze the audio stream for the voice of a medical assistant of healthcare provider 110 who is authorized. In another example, privacy analysis unit 212 may analyze the audio stream for the voices of one or more specific unauthorized people. In some examples, privacy analysis unit 212 may use natural language processing (NLP) techniques to differentiate between authorized and unauthorized people.

Use of the video stream and/or audio stream to determine statuses of privacy aspects of healthcare provider environment 118 may provide specific advantages because it may be unnecessary for healthcare provider computing device 102 to send provide data in addition to normal video and/or audio data of the telemedicine session to telemedicine facilitation application 104 for privacy analysis unit 212 to determine statuses of privacy aspects of healthcare provider environment 118.

Notification unit 214 may be configured to determine, based on a status of a privacy aspect of the environment of a first party of the telemedicine session whether to cause a computing device of a second party of the telemedicine session to present a notification regarding the status of the privacy aspect of the environment of the first party. For instance, notification unit 214 may cause patient computing device 106 to present a notification regarding the status of a privacy aspect of healthcare provider environment 118. In some examples, notification unit 214 may cause healthcare provider computing device 102 to present a notification regarding the status of a privacy aspect of patient environment 120. For ease of explanation, this disclosure generally describes notification unit 214 with respect to causing patient computing device 106 to present a notification regarding a status of a privacy aspect of healthcare provider environment 118, but such description may apply mutatis mutandis with respect to healthcare provider computing device 102 and privacy aspects of patient environment 120.

In some examples where notification unit 214 causes patient computing device 106 to present the notification, the notification may provide information to patient 112 regarding the status of a privacy aspect of healthcare provider environment 118. For example, the notification may indicate to patient 112 that a person other than healthcare provider 110 has entered healthcare provider environment 118. In another example, the notification may indicate to patient 112 that a device with audio or video recording ability is present in healthcare provider environment 118. In some examples, the notification may include user-selectable features that enable patient 112 to continue with the telemedicine session or to notify healthcare provider 110 and pause the telemedicine session. In some examples where notification unit 214 causes patient computing device 106 to present the notification, notification unit 214 may also cause healthcare provider computing device 102 to present a notification regarding the status of the privacy aspect of healthcare provider environment 118.

In some examples, privacy analysis unit 212 may determine that the status of a privacy aspect of healthcare provider environment 118 has changed. For instance, privacy analysis unit 212 may determine that an unauthorized person has entered healthcare provider environment 118. Responsive to privacy analysis unit 212 determining that the status of the privacy aspect of healthcare provider environment 118 has changed, notification unit 214 may cause a user interface of telemedicine facilitation application 104 to present a notification to patient 112. In some examples, notification unit 214 may cause the user interface to include notifications including a list of the insecure privacy aspects of healthcare provider environment 118. This list may be present in the user interface at the beginning of the telemedicine session and, in some examples, may remain in the user interface throughout the telemedicine session. In some examples, notification unit 214 may cause the interface to include notifications regarding statuses of people (e.g., authorized people, unauthorized people, etc.) as being within or outside healthcare provider environment 118.

Stress analysis unit 216 may be configured to determine a stress level of patient 112. In some examples, notification unit 214 may be configured to determine, based on the stress level of patient 112, whether to cause the user interface of telemedicine facilitation application 104 to present a notification to a first party (e.g., patient 112 or healthcare provider 110) of the telemedicine session regarding the status of one or more privacy aspects of the environment of a second party (e.g., patient 112 or healthcare provider 110) of the telemedicine session.

In some examples, the stress level may be expressed as a score. Notification unit 214 may make the determination to cause the user interface of telemedicine facilitation application 104 to present the notification to the first party in response to determining that the score is above a threshold. In some examples, patient 112 and/or healthcare provider 110 may specify the score. For instance, in one example, the score may be expressed on a scale of 1 to 5 and patient 112 or healthcare provider 110 may specify the threshold as 2. Thus, in this example, if the score is 2 or greater, notification unit 214 may cause the user interface to present the notification regarding the status of a privacy aspect of the environment of healthcare provider 110 or patient 112.

In general, patients are more likely to exhibit greater stress when the patients are about to disclose sensitive information to healthcare providers. Moreover, patients are more likely to exhibit greater stress when the patients are aware that healthcare providers are about to disclose sensitive information to the patients. Thus, the stress level of patient 112 may be an indicator that sensitive information is about to be disclosed during a telemedicine session. Determining whether to cause the user interface of telemedicine facilitation application 104 to present a notification based on the stress level of patient 112 may help reduce the number of notifications presented in the user interface of telemedicine facilitation application 104. Reducing the number of notifications presented in the user interface of telemedicine facilitation application 104 may simplify the user interface and generally improve the experience of using telemedicine facilitation application 104.

In some examples, patient data streams 114 include one or more patient biometric data streams. The patient biometric data streams include biometric data regarding one or more biometric markers of patient 112. A wearable device, such as a smartwatch, worn by patient 112 may generate the patient biometric data streams and provide the patient biometric data streams to computing system 108, e.g., via patient computing device 106 or another device. The biometric markers of patient 112 may include heart rate, skin moistness, fidgeting, blood pressure, electrocardiogram patterns, blood volume pulse, skin conductance (e.g., skin conductance level, skin conductance response), and so on. In general, steeper slopes of skin conductance are correlated with stress. Greater heart rate variability is another sign of stress.

In some examples, to determine the stress level of patient 112 based on the biometric data streams, stress analysis unit 216 may obtain baseline measurements of the one or more biometric markers of patient 112. Stress analysis unit 216 may determine a score for individual biometric data streams and may telemedicine facilitation application 104 (e.g., stress analysis unit 216, privacy analysis unit 212, etc.) may determine, based on the scores whether to display a notification when a privacy aspect of the healthcare provider environment changes. In some examples, stress analysis unit 216 may determine an overall score based on the scores for individual biometric data streams (e.g., by totaling the scores for the individual biometric data streams, averaging the scores for the individual biometric data streams, etc.). To determine a score for an individual biometric data stream, stress analysis unit 216 may determine a percentage that a current value of the biometric data stream exceeds a baseline measurement of the biometric data stream. Stress analysis unit 216 may then assign a score for the biometric data stream based on the determined percentage (e.g., each additional 10% above the baseline measurement up to a given limit (e.g., 50%) may correspond to an additional point for the biometric data stream).

As previously described, telemedicine facilitation application 104 may obtain a patient audio stream and a patient video stream from patient computing device 106 during the telemedicine session. Stream forwarding unit 210 of telemedicine facilitation application 104 may provide the patient audio stream and the patient video stream from patient 112 to healthcare provider computing device 102. In some examples, notification unit 214 may analyze the video stream of patient 112 to identify body parts of patient 112 (e.g., specific body parts, general areas of the body of patient 112, groups/systems of body parts of patient 112, etc.) that are represented in the patient video stream. For instance, notification unit 214 may use a machine-learned (ML) image recognition model to identify body parts of patient 112 that are represented in the patient video stream. Based on notification unit 214 identifying a body part that is designated as sensitive, notification unit 214 may cause patient computing device 106 to generate an alert notification if there are unsecure privacy aspects of healthcare provider environment. This may be equivalent to setting the stress threshold to a low value, so that alert notifications are more likely to appear on patient computing device 106 when patient 112 is showing a specific body part. In this way, notification unit 214 may, in effect, assume that patient 112 is exhibiting stress when showing the sensitive body part. Telemedicine facilitation application 104 may receive an indication of which body parts are designated as sensitive from patient 112, healthcare provider 110, or another source.

In some examples, notification unit 214 may analyze a patient audio stream or healthcare provider audio stream for discussion of sensitive body parts. In such examples, based on notification unit 214 determining that discussion of one or more body parts are designated as sensitive is occurring, notification unit 214 may cause patient computing device 106 to generate an alert notification if there are unsecure privacy aspects of healthcare provider environment, even if changes to statuses of such privacy aspects of healthcare provider environment would not otherwise cause notification unit 214 to cause patient computing device 106 to generate an alert notification.

In examples where healthcare provider data streams 116 include a video stream, data hiding unit 218 may analyze the video stream for sensitive information and may obscure the sensitive information. Sensitive information may inadvertently be present in the background of the video stream. For example, data hiding unit 218 may analyze the video stream for sensitive information such as x-rays, patient charts, magnetic resonance imaging (MRI) images, personally identifying information, medical codes, photographs of unauthorized persons, or other types of sensitive information. In some examples, data hiding unit 218 may determine whether the video stream includes screen sharing content that includes sensitive information unrelated to patient 112. Data hiding unit 218 may obscure the sensitive information by modifying the video stream to blur, block out, or otherwise prevent the sensitive information from being seen from the video stream.

Thus, in some examples, when healthcare provider 110 joins a telemedicine session, data hiding unit 218 may analyze a video stream generated by healthcare provider computing device 102 and identify any sensitive information that is visible in the video stream, e.g., using technologies such as image recognition and optical character recognition. When data hiding unit 218 identifies potential sensitive information, data hiding unit 218 may cause healthcare provider computing device 102 to display a notification that notifies healthcare provider 110 that the video stream may include sensitive information. For instance, data hiding unit 218 may invoke an API call of a client of telemedicine facilitation application 104 operating on healthcare provider computing device 102 to request display of a notification to notify healthcare provider 110 that the video stream may include sensitive information. The notification may prompt healthcare provider 110 to remove the sensitive information from view. In some examples, the notification may prompt healthcare provider 110 to indicate whether to continue the telemedicine session. Data hiding unit 218 may obscure the sensitive information if healthcare provider 110 does not remove the sensitive information from view. If new sensitive information enters the video stream after the telemedicine session has started, data hiding unit 218 may obscure the new sensitive information and/or prompt healthcare provider 110 to remove the new sensitive information. In some examples, healthcare provider 110 may want patient 112 to see the sensitive information (e.g., because the sensitive information relates to patient 112). Accordingly, data hiding unit 218 may un-obscure the sensitive information in response to receiving an indication of user input from healthcare provider 110 to un-obscure the sensitive information.

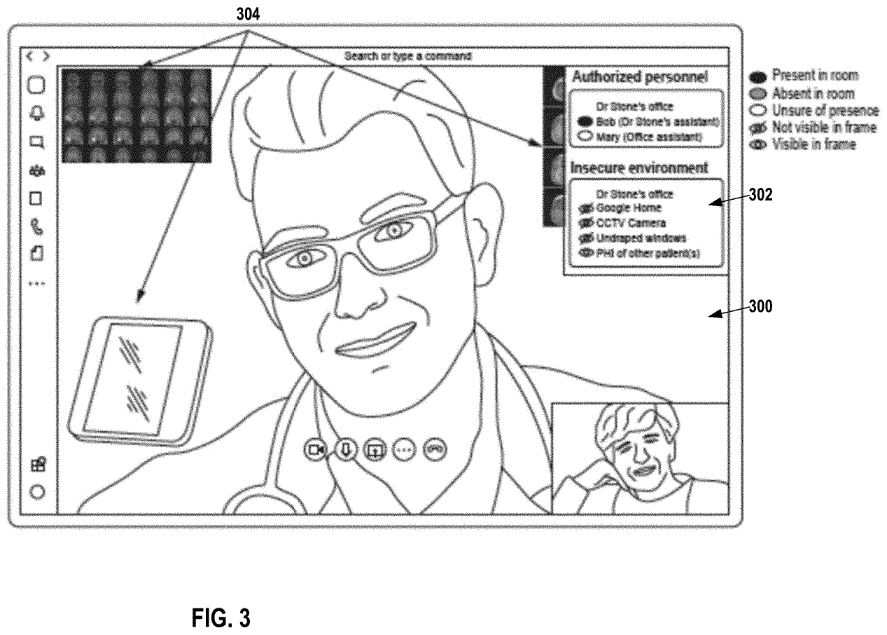

FIG. 3 is a conceptual diagram illustrating example notifications in accordance with one or more aspects of this disclosure. In the example of FIG. 3, patient computing device 106 presents a user interface 300 of telemedicine facilitation application 104. User interface 300 includes video showing healthcare provider 110 and video showing patient 112. User interface 300 may also include notifications 302. Notifications 302 indicate statuses of privacy aspects of healthcare provider environment 118. Specifically, notifications 302 indicate statuses of authorized people in the office of healthcare provider 110. In the example of FIG. 3, healthcare provider 110 is Dr. Stone and the authorized people associated with the office of healthcare provider 110 include “Bob,” who is Dr. Stone's assistant, and “Mary,” who is Dr. Stone's office assistant. The statuses of the authorized people shown in FIG. 3 may be present, absent, or unsure. In this way, notifications 302 may inform patient 112 regarding the status of the authorized people in healthcare provider environment 118.

Additionally, in the example of FIG. 3, notifications 302 indicate privacy aspects of healthcare provider environment 118 that may have insecure statuses. For instance, notifications 302 may indicate that there is a smart speaker device, a closed-circuit television device, and undraped windows in healthcare provider environment 118. Notifications 302 may also indicate whether these insecure privacy aspects are visible or not visible in a frame of the video of healthcare provider 110. Furthermore, notifications 302 may indicate whether sensitive information or non-secure devices 304 is present in healthcare provider environment 118. For instance, MRI images 304 in healthcare provider environment 118 may be sensitive information for another patient.

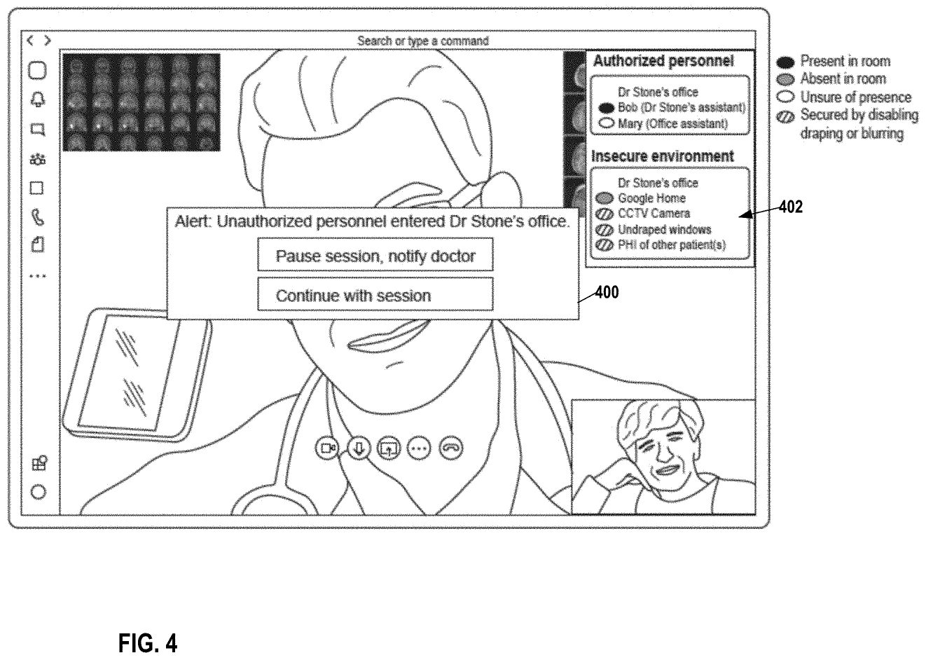

FIG. 4 is a conceptual diagram illustrating example notifications in accordance with one or more aspects of this disclosure. In the example of FIG. 4, notification unit 214 has made a determination to output an alert notification, e.g., in response to a change in a status of a privacy aspect of healthcare provider environment 118. Accordingly, user interface 300 includes an alert notification 400 that informs patient 112 regarding the change in the status of the privacy aspect of healthcare provider environment 118. Alert notification 400 includes a description of the change in the status of the privacy aspect of healthcare provider environment 118. Specifically, in the example of FIG. 4, alert notification 400 indicates that unauthorized personnel have entered Dr. Stone's office. Alert notification 400 includes a first option to pause the telemedicine session and a second option to continue the telemedicine session. In some examples, stream forwarding unit 210 may automatically pause the video stream of patient 112, e.g., until notification unit 214 receives an indication of user input to resume the video stream, until a time limit expires, or one or more other conditions occur. In some examples, when the video stream of patient 112 is paused, stream forwarding unit 210 may instead replace the video stream of patient 112 with a non-sensitive image, such as a non-sensitive image that patient 112 was previously displaying during the telemedicine session.

Furthermore, in the example of FIG. 4, notifications 402 indicate privacy aspects of healthcare provider 110 that have been secured. For instance, notifications 402 indicate that the smart speaker device is absent from a room of healthcare provider environment 118, that the CCTV device has been secured by disabling the device, that the undraped windows have been secured by draping the windows, and the sensitive information has been secured by blurring the sensitive information.

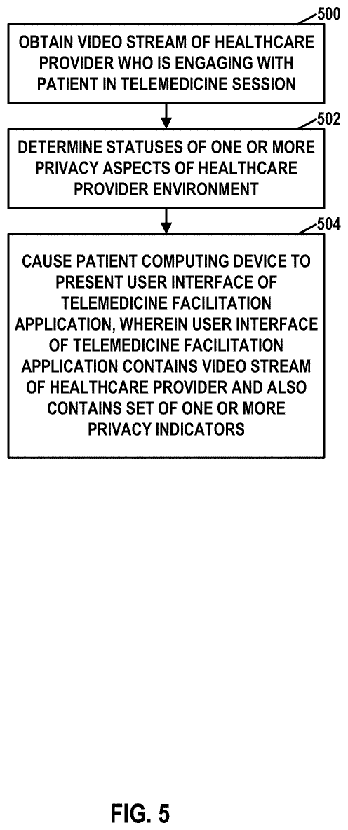

FIG. 5 is a flowchart illustrating an example operation of telemedicine facilitation application 104 in accordance with one or more aspects of this disclosure. The figures of the disclosure are provided as examples. In other examples, operations of telemedicine facilitation application 104 may include more, fewer, or different actions. The flowcharts of this disclosure are described with respect to the other figures of this disclosure. However, the flowcharts of this disclosure are not so limited. For ease of explanation, the flowcharts of this disclosure are described with respect to privacy aspects of healthcare provider environment 118, but the flowchart of this disclosure may be applicable mutatis mutandis with respect to privacy aspects of patient environment 120.

In the example of FIG. 5, telemedicine facilitation application 104 may obtain a video stream of healthcare provider 110, who is engaging with patient 112 in a telemedicine session (500). For example, telemedicine facilitation application 104 may receive the video stream of healthcare provider from healthcare provider computing device 102.

Furthermore, in the example of FIG. 5, telemedicine facilitation application 104 may determine, at the start of and/or during the telemedicine session, statuses of one or more privacy aspects of healthcare provider environment 118 (502). Healthcare provider environment 118 is an environment of healthcare provider 110. The privacy aspects of healthcare provider environment 118 are aspects of healthcare provider environment 118 that have a potential to compromise privacy of sensitive information provided by patient 112 to healthcare provider 110 during the telemedicine session.

As described elsewhere in this disclosure, privacy analysis unit 212 may determine the statuses of the one or more privacy aspects of healthcare provider environment 118 based on one or more healthcare provider data streams 116, such as the video stream of healthcare provider 110, an audio stream of healthcare provider 110, and so on. For example, privacy analysis unit 212 may determine, based at least in part on the video stream of healthcare provider 110, the status of a privacy aspect of healthcare provider environment 118. For instance, in this example, the privacy aspect of healthcare provider environment 118 may relate to the present of nonauthorized personnel in healthcare provider environment 118. Privacy analysis unit 212 may apply a facial recognition system configured to identify faces of people in healthcare provider environment 118 and may determine whether the people in healthcare provider environment 118 are nonauthorized personnel. In some examples where telemedicine facilitation application 104 receives an audio stream of healthcare provider 110, stream forwarding unit 210 of telemedicine facilitation application 104 may cause patient computing device 106 to output the audio stream of healthcare provider 110. Furthermore, in this example, as part of determining the statuses of the one or more privacy aspects of healthcare provider environment 118, privacy analysis unit 212 may determine, based at least in part on the audio stream of the healthcare provider, the status of a specific privacy aspect of healthcare provider environment 118. In some examples, telemedicine facilitation application 104 may obtain, from a computing device of healthcare provider 110 (e.g., healthcare provider computing device 102), data associated with devices or objects in healthcare provider environment 118. In this example, as part of determining the statuses of the one or more privacy aspects of healthcare provider environment 118, privacy analysis unit 212 may determine, based at least in part on the data associated with the device or objects in healthcare provider environment 118, the status of a specific privacy aspect of the healthcare provider environment.

Telemedicine facilitation application 104 may cause patient computing device 106 to present a user interface of telemedicine facilitation application 104 (504), wherein the user interface of telemedicine facilitation application 104 includes the video stream of healthcare provider 110 and also includes a set of one or more notifications, e.g., notifications 302 (FIG. 3), alert notification 400 (FIG. 4), notifications 402 (FIG. 4), etc. Each of the one or more notifications indicates the status of a different one of the privacy aspects of the healthcare provider environment.

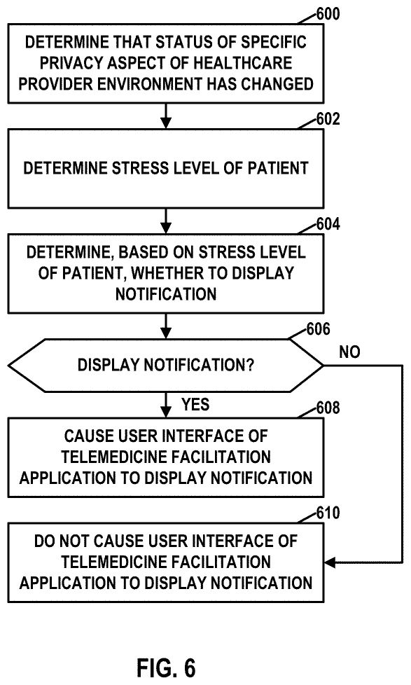

FIG. 6 is a flowchart illustrating an example operation in which notifications are displayed dependent on stress levels, in accordance with one or more aspects of this disclosure. In the example of FIG. 6, privacy analysis unit 212 of telemedicine facilitation application 104 may determine that a status of a specific aspect of healthcare provider environment 118 has changed (600). For example, privacy analysis unit 212 may determine that an unauthorized person is now present in healthcare provider environment 118.

Additionally, stress analysis unit 216 may determine a stress level of patient 112 (602). Stress analysis unit 216 may determine the stress level of patient 112 in accordance with any of the examples provided elsewhere in this disclosure. Notification unit 214 may determine, based on the stress level of patient 112, whether to display a notification (e.g., alert notification 402) regarding the change of status of the specific aspect of healthcare provider environment 118 (604). For instance, notification unit 214 may determine, based on the stress level of patient 112 being above a threshold, to display the notification regarding the change of status of the specific aspect of healthcare provider environment 118. In other words, notification unit 214 may make a determination to cause the user interface of telemedicine facilitation application 104 to display the notification based on the stress level of being above the threshold. In some examples, the stress level of patient 112 may be one of a plurality of inputs to a machine-learned model that notification unit 214 uses to determine whether to display the notification regarding the change of status of the specific aspect of healthcare provider environment 118.

Based on a determination to display the notification (“YES” branch of 606), notification unit 214 may cause a user interface of telemedicine facilitation application 104 to display the notification regarding the change of status of the specific aspect of healthcare provider environment 118 (608). For example, notification unit 214 may invoke a method of an API implemented by a client of telemedicine facilitation application 104 operating on patient computing device 106 to cause a user interface of telemedicine facilitation application 104 shown on patient computing device 106 to display the notification. In some examples, notification unit 214 may modify a video stream sent to and displayed on patient computing device 106 to include the notification. On the other hand, based on a determination not to display the notification (“NO” branch of 606), notification unit 213 does not cause the user interface of telemedicine facilitation application 104 to display the notification (610).

The following is a non-limiting list of aspects that are in accordance with one or more techniques of this disclosure.

Aspect 1: A method includes obtaining, by a computing system, a video stream of a first party who is engaging with a second party in a telemedicine session; determining, by the computing system, at the start of and/or during the telemedicine session, statuses of one or more privacy aspects of a first party environment, wherein the first party environment is an environment of the first party, wherein the privacy aspects of the first party environment are aspects of the first party environment that have a potential to compromise privacy of sensitive information provided by the second party to the first party during the telemedicine session; and causing, by the computing system, a second party computing device to present a user interface of a telemedicine facilitation application, wherein the user interface of the telemedicine facilitation application includes the video stream of the first party and also includes a set of one or more notifications, wherein each of the one or more notifications indicates the status of a different one of the privacy aspects of the first party environment.

Aspect 2: The method of aspect 1, further includes determining, by the computing system, that the status of a specific privacy aspect of the first party environment has changed; and responsive to determining that the status of the specific privacy aspect of the first party environment has changed, causing, by the computing system, the user interface of the telemedicine facilitation application to present a notification indicating the status of the specific privacy aspect.

Aspect 3: The method of aspect 2, further includes determining, by the computing system, a stress level of the second party; and determining, by the computing system, based on the stress level of the second party, whether to cause the user interface of the telemedicine facilitation application to present the notification.

Aspect 4: The method of aspect 3, wherein determining whether to cause the user interface of the telemedicine facilitation application to display the notification comprises: determining, by the computing system, whether the stress level of the second party is above a threshold; and making a determination, by the computing system, to cause the user interface of the telemedicine facilitation application to display the notification based on the stress level being above the threshold.

Aspect 5: The method of any of aspects 2 through 4, wherein the notification includes a first option to pause the telemedicine session and a second option to continue the telemedicine session.

Aspect 6: The method of any of aspects 1 through 5, wherein determining the statuses of the one or more privacy aspects of the first party environment comprises determining, by the computing system, based at least in part on the video stream of the first party, the status of a specific privacy aspect of the first party environment.

Aspect 7: The method of aspect 6, wherein the specific privacy aspect of the first party environment relates to presence of nonauthorized personnel in the first party environment, and wherein determining the status of the specific privacy aspect of the first party environment comprises: applying, by the computing system, a facial recognition system configured to identify faces of people in the first party environment; and determining, by the computing system, whether the people in the first party environment are nonauthorized personnel.

Aspect 8: The method of any of aspects 1 through 7, wherein the method further comprises: obtaining, by the computing system, an audio stream of the first party; and causing, by the computing system, the second party computing device to output the audio stream of the first party, and wherein determining the statuses of the one or more privacy aspects of the first party environment comprises determining, by the computing system, based at least in part on the audio stream of the first party, the status of a specific privacy aspect of the first party environment.

Aspect 9: The method of any of aspects 1 through 8, wherein the method further comprises obtaining, by the computing system, from a computing device of the first party, data associated with devices or objects in the first party environment; and wherein determining the statuses of the one or more privacy aspects of the first party environment comprises determining, by the computing system, based at least in part on the data associated with the devices or objects in first party environment, the status of a specific privacy aspect of the first party environment.

Aspect 10: The method of any of aspects 1 through 9, wherein the first party is a healthcare provider, and the second party is a patient.

Aspect 11: A computing system includes a communication unit configured to obtain a video stream of a first party who is engaging with a second party in a telemedicine session; and one or more processors implemented in circuitry and in communication with the memory, the one or more processors configured to: determine, at the start of and/or during the telemedicine session, statuses of one or more privacy aspects of a first party environment, wherein the first party environment is an environment of the first party, wherein the privacy aspects of the first party environment are aspects of the first party environment that have a potential to compromise privacy of sensitive information provided by the second party to the first party during the telemedicine session; and cause a second party computing device to present a user interface of a telemedicine facilitation application, wherein the user interface of the telemedicine facilitation application includes the video stream of the first party and also includes a set of one or more notifications, wherein each of the one or more notifications indicates the status of a different one of the privacy aspects of the first party environment.

Aspect 12: The computing system of aspect 11, wherein the one or more processors are further configured to: determine that the status of a specific privacy aspect of the first party environment has changed; and responsive to determining that the status of the specific privacy aspect of the first party environment has changed, cause the user interface of the telemedicine facilitation application to present a notification indicating the status of the specific privacy aspect.

Aspect 13: The computing system of aspect 12, wherein the one or more processors are further configured to: determine a stress level of the second party; and determine, based on the stress level of the second party, whether to cause the user interface of the telemedicine facilitation application to present the notification.

Aspect 14: The computing system of aspect 13, wherein the one or more processors are configured to, as part of determining whether to cause the user interface of the telemedicine facilitation application to display the notification: determine whether the stress level of the second party is above a threshold; and make a determination to cause the user interface of the telemedicine facilitation application to display the notification based on the stress level being above the threshold.

Aspect 15: The computing system of any of aspects 12 through 14, wherein the notification includes a first option to pause the telemedicine session and a second option to continue the telemedicine session.

Aspect 16: The computing system of any of aspects 11 through 15, wherein the one or more processors are configured to, as part of determining the statuses of the one or more privacy aspects of the first party environment, determine, based at least in part on the video stream of the first party, the status of a specific privacy aspect of the first party environment.

Aspect 17: The computing system of aspect 16, wherein the specific privacy aspect of the first party environment relates to presence of nonauthorized personnel in the first party environment, and wherein the one or more processors are configured to, as part of determining the status of the specific privacy aspect of the first party environment: apply a facial recognition system configured to identify faces of people in the first party environment; and determine whether the people in the first party environment are nonauthorized personnel.

Aspect 18. The computing system of any of aspects 11-17, wherein the one or more processors are further configured to: obtain an audio stream of the first party; and cause the second party computing device to output the audio stream of the first party, and wherein the one or more processors are configured to, as part of determining the statuses of the one or more privacy aspects of the first party environment, determine, based at least in part on the audio stream of the first party, the status of a specific privacy aspect of the first party environment.

Aspect 19: The computing system of any of aspects 11 through 17, wherein the one or more processors are further configured to obtain, from a computing device of the first party, data associated with devices or objects in the first party environment; and wherein the one or more processors are configured to, as part of determining the statuses of the one or more privacy aspects of the first party environment, determine, based at least in part on the data associated with the devices or objects in first party environment, the status of a specific privacy aspect of the first party environment.

Aspect 20: A non-transitory computer-readable medium having instructions stored thereon that, when executed, cause one or more processors to: obtain a video stream of a first party who is engaging with a second party in a telemedicine session; determine, at the start of and/or during the telemedicine session, statuses of one or more privacy aspects of a first party environment, wherein the first party environment is an environment of the first party, wherein the privacy aspects of the first party environment are aspects of the first party environment that have a potential to compromise privacy of sensitive information provided by the second party to the first party during the telemedicine session; and cause a second party computing device to present a user interface of a telemedicine facilitation application, wherein the user interface of the telemedicine facilitation application includes the video stream of the first party and also includes a set of one or more notifications, wherein each of the one or more notifications indicates the status of a different one of the privacy aspects of the first party environment.

For processes, apparatuses, and other examples or illustrations described herein, including in any flowcharts or flow diagrams, certain operations, acts, steps, or events included in any of the techniques described herein can be performed in a different sequence, may be added, merged, or left out altogether (e.g., not all described acts or events are necessary for the practice of the techniques). Moreover, in certain examples, operations, acts, steps, or events may be performed concurrently, e.g., through multi-threaded processing, interrupt processing, or multiple processors, rather than sequentially. Further certain operations, acts, steps, or events may be performed automatically even if not specifically identified as being performed automatically. Also, certain operations, acts, steps, or events described as being performed automatically may be alternatively not performed automatically, but rather, such operations, acts, steps, or events may be, in some examples, performed in response to input or another event.

Further, certain operations, techniques, features, and/or functions may be described herein as being performed by specific components, devices, and/or modules. In other examples, such operations, techniques, features, and/or functions may be performed by different components, devices, or modules. Accordingly, some operations, techniques, features, and/or functions that may be described herein as being attributed to one or more components, devices, or modules may, in other examples, be attributed to other components, devices, and/or modules, even if not specifically described herein in such a manner.

In one or more examples, the functions described may be implemented in hardware, software, firmware, or any combination thereof. If implemented in software, the functions may be stored on or transmitted over a computer-readable medium as one or more instructions or code and executed by a hardware-based processing unit. Computer-readable media may include computer-readable storage media, which corresponds to a tangible medium such as data storage media, or communication media including any medium that facilitates transfer of a computer program from one place to another, e.g., according to a communication protocol. In this manner, computer-readable media generally may correspond to (1) tangible computer-readable storage media which is non-transitory or (2) a communication medium such as a signal or carrier wave. Data storage media may be any available media that can be accessed by one or more computers, processing circuitry, or one or more processors to retrieve instructions, code and/or data structures for implementation of the techniques described in this disclosure. A computer program product may include a computer-readable medium.

By way of example, and not limitation, such computer-readable storage media can include RAM, ROM, EEPROM, CD-ROM, or other optical disk storage, magnetic disk storage, or other magnetic storage devices, flash memory, or any other medium that can be used to store desired program code in the form of instructions or data structures and that can be accessed by a computer. Also, any connection is properly termed a computer-readable medium. For example, if instructions are transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio, and microwave are included in the definition of medium. It should be understood, however, that computer-readable storage media and data storage media do not include connections, carrier waves, signals, or other transitory media, but are instead directed to non-transitory, tangible storage media. Disk and disc, as used herein, includes compact disc (CD), laser disc, optical disc, digital versatile disc (DVD), and Blu-ray disc, where disks usually reproduce data magnetically, while discs reproduce data optically with lasers. Combinations of the above should also be included within the scope of computer-readable media.

Instructions may be executed by processing circuitry (e.g., one or more processors, such as one or more DSPs, general purpose microprocessors, ASICs, FPGAs, or other equivalent integrated or discrete logic circuitry), as well as any combination of such components. Accordingly, the term “processor” or “processing circuitry” as used herein, may refer to any of the foregoing structures or any other structure suitable for implementation of the techniques described herein. In addition, in some aspects, the functionality described herein may be provided within dedicated hardware and/or software modules. Also, the techniques could be fully implemented in one or more circuits or logic elements.

The techniques of this disclosure may be implemented in a wide variety of devices or apparatuses, including a wireless communication device or wireless handset, a microprocessor, an integrated circuit (IC) or a set of ICs (e.g., a chip set). Various components, modules, or units are described in this disclosure to emphasize functional aspects of devices configured to perform the disclosed techniques, but do not necessarily require realization by different hardware units. Rather, as described above, various units may be combined in a hardware unit or provided by a collection of interoperative hardware units, including one or more processors as described above, in conjunction with suitable software and/or firmware.

What is claimed is:

1. A method comprising:
obtaining, by a computing system, a video stream of a first party who is engaging with a second party in a telemedicine session;
determining, by the computing system, at the start of and/or during the telemedicine session, statuses of one or more privacy aspects of a first party environment, wherein the first party environment is an environment of the first party, wherein the privacy aspects of the first party environment are aspects of the first party environment that have a potential to compromise privacy of sensitive information provided by the second party to the first party during the telemedicine session; and
causing, by the computing system, a second party computing device to present a user interface of a telemedicine facilitation application, wherein the user interface of the telemedicine facilitation application includes the video stream of the first party and also includes a set of one or more notifications, wherein each of the one or more notifications indicates the status of a different one of the privacy aspects of the first party environment.

2. The method of claim 1, further comprising:
determining, by the computing system, that the status of a specific privacy aspect of the first party environment has changed; and
responsive to determining that the status of the specific privacy aspect of the first party environment has changed, causing, by the computing system, the user interface of the telemedicine facilitation application to present a notification indicating the status of the specific privacy aspect.

3. The method of claim 2, further comprising:
determining, by the computing system, a stress level of the second party; and
determining, by the computing system, based on the stress level of the second party, whether to cause the user interface of the telemedicine facilitation application to present the notification.

4. The method of claim 3, wherein determining whether to cause the user interface of the telemedicine facilitation application to display the notification comprises:
determining, by the computing system, whether the stress level of the second party is above a threshold; and
making a determination, by the computing system, to cause the user interface of the telemedicine facilitation application to display the notification based on the stress level being above the threshold.

5. The method of claim 2, wherein the notification includes a first option to pause the telemedicine session and a second option to continue the telemedicine session.

6. The method of claim 1, wherein determining the statuses of the one or more privacy aspects of the first party environment comprises determining, by the computing system, based at least in part on the video stream of the first party, the status of a specific privacy aspect of the first party environment.

7. The method of claim 6,
wherein the specific privacy aspect of the first party environment relates to presence of nonauthorized personnel in the first party environment, and
wherein determining the status of the specific privacy aspect of the first party environment comprises:
applying, by the computing system, a facial recognition system configured to identify faces of people in the first party environment; and
determining, by the computing system, whether the people in the first party environment are nonauthorized personnel.

8. The method of claim 1,
wherein the method further comprises:
obtaining, by the computing system, an audio stream of the first party; and
causing, by the computing system, the second party computing device to output the audio stream of the first party, and
wherein determining the statuses of the one or more privacy aspects of the first party environment comprises determining, by the computing system, based at least in part on the audio stream of the first party, the status of a specific privacy aspect of the first party environment.

9. The method of claim 1,
wherein the method further comprises obtaining, by the computing system, from a computing device of the first party, data associated with devices or objects in the first party environment; and
wherein determining the statuses of the one or more privacy aspects of the first party environment comprises determining, by the computing system, based at least in part on the data associated with the devices or objects in first party environment, the status of a specific privacy aspect of the first party environment.

10. The method of claim 1, wherein the first party is a healthcare provider, and the second party is a patient.

11. A computing system comprising:
a communication unit configured to obtain a video stream of a first party who is engaging with a second party in a telemedicine session; and
one or more processors implemented in circuitry and in communication with the memory, the one or more processors configured to:
determine, at the start of and/or during the telemedicine session, statuses of one or more privacy aspects of a first party environment, wherein the first party environment is an environment of the first party, wherein the privacy aspects of the first party environment are aspects of the first party environment that have a potential to compromise privacy of sensitive information provided by the second party to the first party during the telemedicine session; and
cause a second party computing device to present a user interface of a telemedicine facilitation application, wherein the user interface of the telemedicine facilitation application includes the video stream of the first party and also includes a set of one or more notifications, wherein each of the one or more notifications indicates the status of a different one of the privacy aspects of the first party environment.

12. The computing system of claim 11, wherein the one or more processors are further configured to:
determine that the status of a specific privacy aspect of the first party environment has changed; and
responsive to determining that the status of the specific privacy aspect of the first party environment has changed, cause the user interface of the telemedicine facilitation application to present a notification indicating the status of the specific privacy aspect.

13. The computing system of claim 12, wherein the one or more processors are further configured to:
determine a stress level of the second party; and
determine, based on the stress level of the second party, whether to cause the user interface of the telemedicine facilitation application to present the notification.

14. The computing system of claim 13, wherein the one or more processors are configured to, as part of determining whether to cause the user interface of the telemedicine facilitation application to display the notification:
determine whether the stress level of the second party is above a threshold; and
make a determination to cause the user interface of the telemedicine facilitation application to display the notification based on the stress level being above the threshold.

15. The computing system of claim 12, wherein the notification includes a first option to pause the telemedicine session and a second option to continue the telemedicine session.

16. The computing system of claim 11, wherein the one or more processors are configured to, as part of determining the statuses of the one or more privacy aspects of the first party environment, determine, based at least in part on the video stream of the first party, the status of a specific privacy aspect of the first party environment.

17. The computing system of claim 16, wherein the specific privacy aspect of the first party environment relates to presence of nonauthorized personnel in the first party environment, and wherein the one or more processors are configured to, as part of determining the status of the specific privacy aspect of the first party environment:
apply a facial recognition system configured to identify faces of people in the first party environment; and
determine whether the people in the first party environment are nonauthorized personnel.

18. The computing system of claim 11,
wherein the one or more processors are further configured to:
obtain an audio stream of the first party; and
cause the second party computing device to output the audio stream of the first party, and
wherein the one or more processors are configured to, as part of determining the statuses of the one or more privacy aspects of the first party environment, determine, based at least in part on the audio stream of the first party, the status of a specific privacy aspect of the first party environment.

19. The computing system of claim 11,
wherein the one or more processors are further configured to obtain, from a computing device of the first party, data associated with devices or objects in the first party environment; and
wherein the one or more processors are configured to, as part of determining the statuses of the one or more privacy aspects of the first party environment, determine, based at least in part on the data associated with the devices or objects in first party environment, the status of a specific privacy aspect of the first party environment.

20. A non-transitory computer-readable medium having instructions stored thereon that, when executed, cause one or more processors to:
obtain a video stream of a first party who is engaging with a second party in a telemedicine session;
determine, at the start of and/or during the telemedicine session, statuses of one or more privacy aspects of a first party environment, wherein the first party environment is an environment of the first party, wherein the privacy aspects of the first party environment are aspects of the first party environment that have a potential to compromise privacy of sensitive information provided by the second party to the first party during the telemedicine session; and
cause a second party computing device to present a user interface of a telemedicine facilitation application, wherein the user interface of the telemedicine facilitation application includes the video stream of the first party and also includes a set of one or more notifications, wherein each of the one or more notifications indicates the status of a different one of the privacy aspects of the first party environment.
